(19) United States
(12) Patent Application Publication
(10) Pub. No.: US 2005/0010449 A1
(43) Pub. Date: Jan. 13, 2005

(54) Living and deceased human organ exchange program

(76) Inventor: Loay K. Abukwedar, Columbia, MD (US)

Correspondence Address: William S. Ramsey, 5253 Even Star Place, Columbia, MD, 21044, US

(21) Appl. No.: 10/817,635

(22) Filed: Apr. 2, 2004

Related U.S. Application Data
(63) Continuation-in-part of application No. 10/616,838, filed on Jul. 10, 2003.

(51) Int. Cl. G06F 17/60

(52) U.S. Cl. 705/3

(57) ABSTRACT

This invention is a registry for humans in need of an organ transplantation who, in exchange for an organ or part of an organ which meets their needs, are willing to donate an organ or part of an organ for transplantation to another human in need of an organ transplantation. The exchange may be direct between two individuals, for example between a first patient in need of a kidney who donates a lung lobe to a second patient in need of a lung lobe, while the second patient donates a kidney to the first patient. In another embodiment, the exchange is indirect and involves greater than two patients, for example, the donation of a kidney by a first patient to a second patient, the donation of a liver lobe by the second patient to a third patient, and the donation of a lung lobe by the third patient to the first patient. If a person in the registry is unable to donate an organ while alive, the donation will occur after the patient's death. This registry insures the maximum availability of organ donations by living persons.

### CROSS-REFERENCE TO RELATED APPLICATIONS

This is a continuation-in-part application of Ser. No. 10/616,838, filed Jul. 10, 2003, hereby abandoned.

### STATEMENT REGARDING FEDERALLY SPONSORED RESEARCH OR DEVELOPMENT

Not Applicable

### REFERENCE TO A “MICROFICHE APPENDIX”

Not Applicable.

### BACKGROUND OF THE INVENTION

This invention relates to increasing the supply of human organs for transplantation.

### FIELD OF THE INVENTION

### Description of Related Art Including Information Disclosed Under 37 CFR 1.97 and 37 CFR 1.98

This patent application discloses an improved method of increasing the supply of human organs for transplantation, the Living and Deceased Human Organ Exchange Program (PROGRAM).

Many lives could be saved, and vast suffering relieved, if the supply of human organs were increased to approximate the need for such tissues. Thousands of patients are on waiting lists for organs; a substantial number of such patients die each year. There are only two sources for human organs, living and deceased donors. There is general agreement that organs from living donors provide better results than those from deceased donors.

This invention is a process which pairs two persons from a donor-recipient registry who have compatible parameters for transplantation, each of whom are in need of transplanted organs or portions of organs. A transplantation donor for a specific organ or portion of an organ would act as a transplantation recipient for a different organ or portion of an organ. The process can be extended to three or more subjects. This invention will extend to all patients who need organ transplantations the opportunity to exchange with other patients all or part of a healthy organ they have for all or part of a healthy organ they need.

The right of a member of the donor-recipient registry who donated an organ to receive a needed organ would extend to suitable organs from deceased members of the registry. The morbidity and mortality of members of the registry, certainly relieved by the receipt of needed organs, nevertheless would prevent some of the members from becoming living organ donors. These members, after death, would become organ donors for other members of the registry who were living donors.

U.S. Pat. No. 4,491,725 discloses a system which reads a medical information card to determine and utilize the patient's background medical and insurance information. A central database is used to determine the insurance payments.

U.S. Pat. No. 5,993,387 discloses a registry for placental and umbilical cord stem cells. A central database is used to store information on cord blood. The cells are stored for potential use by the child, the family of the child, or unrelated matched recipients.

U.S. Pat. No. 6,151,589 discloses a continuous auction system for a computer network (internet) with provisions for data concerning the type, number of items, minimum bids, and time parameters used to determine the optimum time for closing the auction and starting the next auction.

Published U.S. Pat. Application No. US 2002/0049642 discloses a system in which a customer enters data on a product and the database is accessed for matching products, and the information is transmitted to the customer.

The Organ Procurement and Transplantation Network has developed policies covering the donation and receipt of human organs. Policy 3 at 3.5.11.6 provides the assignment of 4 points to a candidate recipient for a kidney transportation to patients which have donated for transplantation in the U.S. his or her vital organs or a segment of a vital organ (i.e., kidney, liver segment, lung segment, partial pancreas, small bowel segment). This raises the priority of a person on a waiting list for a kidney if that person has donated a vital organ. Organ Procurement and Transplantation Network, Policy 3 [online], Organ Distribution, 3.5.11.6, Donation Status, pages 3-21 to 3.22, Nov. 15, 2002 [retrieved on Jun. 16, 2003]. Retrieved from www.optn.org.

A living donor and cadaveric exchange program has been initiated. In this program, a mother, who was not biologically compatible with her son, donated a kidney to a stranger, thus allowing her 13-year-old son to move to the top of the waiting list for a cadaveric kidney transplant. Another variation is called “Paired Exchange.” In a paired exchange there is a first family with a member who is willing but unable to donate a kidney to a first family member in need. This first pair seeks out a similar second pair in which the first donor is compatible with the second pair recipient and the second pair donor is compatible with the first pair recipient. There then is an exchange of kidneys in which all participants are living. Linda Ohler, Expanding the Donor Pool with Living Donors. Progress in Transplantation, Vol. 11, No. 3, 2001, pages 160-161 [retrieved on Jun. 26, 2003]. Retrieved from www.medscape.com/viewarticle/414080.

None of the prior art disclosures fulfill the objectives of the present patent application, that of increasing the supply of human organs and partial organs through a process of pairing persons in need of an organ transplantation with the donation of one organ and receipt of another organ by each person in need of an organ. Such persons are placed in a donor-recipient registry; access by a living donor to a suitable organ from a member of the registry would include organs from deceased members of the registry.

### BRIEF SUMMARY OF THE INVENTION

This invention is a method for creating and maintaining a Living and Deceased Human Organ Exchange Program (PROGRAM). The PROGRAM is for use of a first patient in need of a human organ or part of an organ transplantation who is willing and able to donate a different organ or portion of an organ to a second patient in exchange for the organ or part of an organ for which the first person has a need which is donated by the second patient (each patient is termed a “donor-recipient”). In a second embodiment, the exchange is not direct between two donor-recipients but indirect, between three or more donor-recipients. The invention comprises the steps of creating a new donor-recipient record for a potential donor-recipient comprising compatibility parameters, and matching criteria including the needed organ or part of organ, and organ or part of organ which can be donated. The donor-recipient record is stored in a database or registry for access and searching by other donor-recipients. The donor-recipient records in the database are modified as the donor-recipient's circumstances change. In a third embodiment, a member of the donor-recipient registry who donates an organ to another member obtains access to a different organ from either a living or deceased member of the registry.

This invention includes the process of exchanging a donatable human organ between a living first person in need of a human donatable organ transplant and a living second person in need of a human donatable organ transplant who is willing to donate an organ to the first person, or between the first person and a deceased third person who, while living, was willing to donate an organ to the first person. The first step includes forming a donor-recipient registry of persons in need of a donatable human organ who are willing to donate a different human donatable organ to another person on the registry while the donor is alive or after death of the donor. The second step includes transplanting a donatable human organ to a living first person who is listed on the registry from a living second person who is listed on the registry or from a deceased third person who is listed on the registry.

One objective of this invention is to match, facilitate and arrange the organ exchange between organ transplantation candidates over the world.

Another objective of this invention is to relieve the suffering and mortality of patients in need of organ transplantation by increasing the supply of organs for transplantation.

Another objective is to reduce the cost associated with living donor organ donation.

Another objective is to reduce the cost associated with the treatment of patients on the waiting list for an organ transplantation.

Another objective is to create a database or registry containing donor-recipient records and in which donor-recipient patients who are in need of a donatable human organ and who are willing to donate a donatable human organ while alive or after death.

Another objective is to create a donor-recipient registry of persons to which a member of the registry may donate a donated organ and have access to a different organ donated by a living or deceased member of the registry.

A final objective is to facilitate the transplantation of human organs or parts of organs to patients in need without adverse effects on the environment.

### BRIEF DESCRIPTION OF THE SEVERAL VIEWS OF THE DRAWING

FIG. 1 is a diagrammatic depiction of an exchange of organs between two patients.

FIG. 2 is a diagrammatic depiction of an exchange of organs between three patients.

FIG. 3 is a diagrammatic depiction of an exchange of organs between more than three patients.

FIG. 4 is a diagrammatic depiction of the network to be used to implement the PROGRAM.

FIG. 5 is a flow chart of the processes to be used in creating a donor-recipient record, entering the record into the database, and searching the database for matches.

FIG. 6 is a flow chart of the processes to be used in updating the PROGRAM database after a suitable exchange has been identified by a search of the database.

FIG. 7 is a flow chart of the processes to be used in updating the PROGRAM database when no suitable exchange has been identified by a search of the database.

FIG. 8 is a flow chart of the relationships between users at a transplantation center, PROGRAM software, and the database.

FIG. 9 is a flow chart showing the flow of information between files comprising the registry in the PROGRAM database and between the transplantation centers.

FIG. 10 is a diagrammatic depiction of an exchange of organs between a living donor and a recipient followed by receipt of an organ from the recipient after death.

FIG. 11 is a diagrammatic depiction of an exchange of organs between a living donor and a recipient followed by receipt of an organ from a different member of the donor-recipient registry after death.

### DETAILED DESCRIPTION OF THE INVENTION

A number of human organs can be donated by living donors without significant adverse effect on the donor. For example, a single kidney can be donated with little risk to the donor. The remaining kidney compensates to provide the function of both kidneys. A living donor can donate a segment of the liver. The liver has the capacity to regenerate the segment that was removed and the liver regains full function. Living donors can donate a lobe of the lung. Although lung lobes do not regenerate, the lack of a single lobe does not have a significant effect on the donor. The donation of a single lobe of the lung can be life-saving to a recipient in need of such a donation. Similarly, living individuals can donate a portion of the pancreas. Like the lung, the pancreas does not regenerate but living donors usually have no problems with reduced pancreas function. Similarly, the donation of a small bowel segment does not entail significant risk in function to a living donor. Blood and bone marrow can be donated by living donors without adverse effect. It will be expected that medical science will expand the list of organs and body parts that can be donated by living donors.

In this patent application, each patient listed in a registry, termed a “donor-recipient registry,” in which each patient listed on the registry is in need of a human organ or part of an organ transplantation and is willing and able to donate a different organ or portion of an organ to another patient listed on the registry, the donation to take place while the donor is living or after death of the donor. Each patient involved is termed a “donor-recipient.” An organ or part of an organ are termed “organ.” An organs or part of an organ which can be donated without substantial and lasting adverse effect on the donor are termed “donatable organs.” A database of donor-recipients which store the data and can be searched for matches between donor-recipients is termed a “registry.” The collection of donor-recipients also is termed a “registry.”

The age of an organ donor is not a preventive factor in donation, except to the degree that the size of the organ to be donated is a factor of age. Only a very few medical conditions preclude organ donation, and it is to be expected that progress in medical science will reduce the number of persons unable to donate organs.

Participants in the database of this invention would be paired according to “compatibility parameters” and “matching criteria.” The primary “compatibility parameter” is blood type. Other compatibility parameters include HLA −A, B, and DR antigens, cross-matching, and PRA status. Other compatibility parameters vary according to the organs involved. It is to be expected that medical science will expand or contract the compatibility parameters in the future. In addition, it is possible that the compatibility parameters used in the PROGRAM of this invention may differ from compatibility parameters used in other programs.

The “matching criteria” include the type and size of the organs needed and those which could be donated. Some other matching criteria may be involved, including the medical urgency of the recipient, the degree of immune-system match between donor and recipient, and whether the recipient is a child or an adult. Other matching criteria include age, sex, insurance and other financial information, the urgency and preferred time limit for completing the exchange, and the patient's health status. It is to be expected that medical science will expand or contract the matching criteria in the future. In addition, it is possible that the matching criteria used in the PROGRAM of this invention may differ from the matching criteria used in other programs.

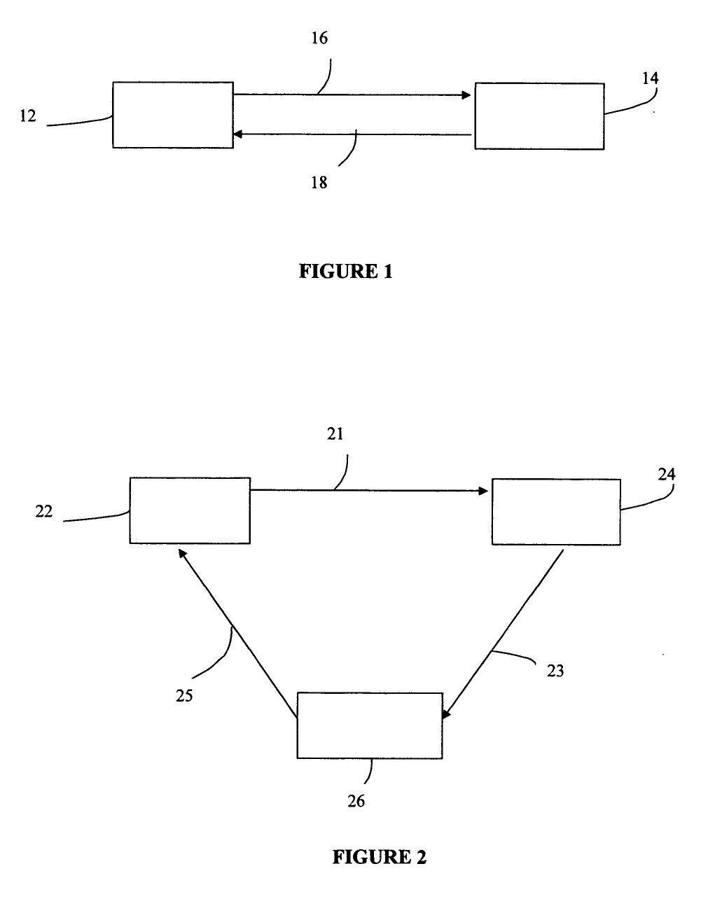

FIG. 1 is a diagrammatic depiction of an exchange of donatable organs between two patients. In FIG. 1, a first patient 12 has need of a kidney and is capable of donating other donatable organs. A second patient 14 has need of a lung lobe but is capable of donating other donatable organs including a kidney. Each patient has a need for a different donatable organ. Both patients have similar compatibility parameters and matching criteria which indicate good prospects for an exchange of organs. Step 16 indicates the donation of a lung lobe by the first patient 12 to the second patient 14 in a transplantation process. Step 18 indicates the donation of a kidney by the second patient 14 to the first patient 12 in a transplantation process. Each of the patients must undergo two operations, the operation to remove the organ to be donated, and the operation to receive the organ for which the patient has a need. Both operations to donate and receive an organ could be done for both patients at the same time or one operation could be delayed, according to the individual medical needs of the patients as determined at the individual transplant centers. Although the Example in FIG. 1 involves the donation and receipt of a kidney and lung lobe, any donatable organs may be used. The order of the transplantation operations is dictated by medical considerations and is not important to the process of the invention.

FIG. 2 is a diagrammatic depiction of an exchange of organs between three patients. In FIG. 2, a first patient 22 has need of a kidney and is capable of donating other donatable organs. A second patient 24 has need of a lung lobe but is capable of donating other donatable organs. A third patient 26 has need of a liver lobe but is capable of donating other donatable organs. All three patients have similar compatibility parameters and matching criteria which indicate good prospects for an exchange of organs. Step 21 indicates the donation of a lung lobe by the first patient 22 to the second patient 24 in a transplantation process. Step 23 indicates the donation of a liver lobe by the second patient 24 to the third patient 26 in a transplantation process. Step 25 indicates the donation of a kidney by the third patient 26 to the first patient 22 in a transplantation process. Each one of the patients must undergo two operations, the operation to remove the organ to be donated, and the operation to receive the organ for which the patient has a need. Both operations to donate and receive an organ could be done for all patients at the same time or one or all operation(s) could be delayed, according to the individual medical needs of the patients as determined at the individual transplant centers. Although the Example in FIG. 2 involves the donation and receipt of a kidney, lung lobe, and liver lobe, any donatable organs may be used. The order of the transplantation operations is dictated by medical considerations and is not important to the process of the invention.

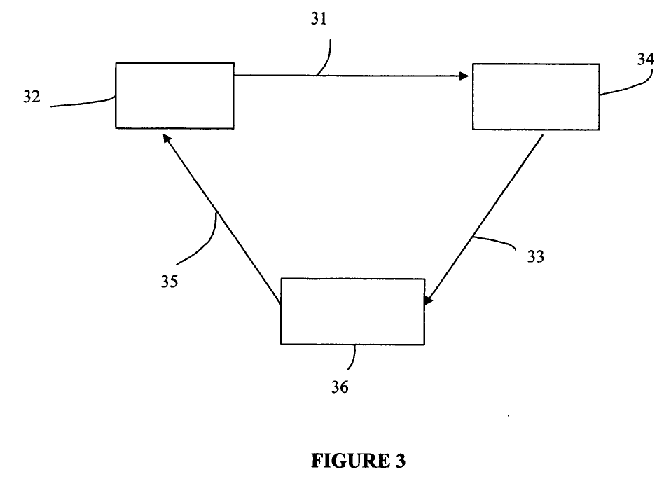

FIG. 3 is a diagrammatic depiction of an exchange of organs between more than three patients. In FIG. 3, a first patient 32 has need of a kidney and is capable of donating other donatable organs. A second patient 34 has need of a lung lobe but is capable of donating other donatable organs. One of N patients, with N greater than two, has need of a liver lobe but is capable of donating other donatable organs. Additional patients, indicated as N patients, have need of donatable organs and are capable of donating other donatable organs. All patients have similar compatibility parameters and matching criteria which indicate good prospects for an exchange of organs. Step 31 indicates the donation of a lung lobe by the first patient 32 to the second patient 34 in a transplantation process. Step 33 indicates the donation of a liver lobe by the second patient 34 to the first of N patients 36 in a transplantation process. Additional transplantation steps involving the N patients are analogous to the steps in FIG. 2 but are not shown in FIG. 3. Step 35 indicates the donation of a kidney by the last patient of N patients 36 to the first patient 32 in a transplantation process. Each one of the patients must undergo two operations, the operation to remove the organ to be donated, and the operation to receive the organ for which the patient has a need. Although the Example in FIG. 3 involves the donation and receipt of a kidney, lung lobe, and liver lobe, any donatable organs may be used. Both operations to donate and receive an organ could be done for all patients at the same time or one or more operation(s) could be delayed, according to the individual medical needs of the patients as determined at the individual transplant centers. The order of the transplantation operations is dictated by medical considerations and is not important to the process of the invention.

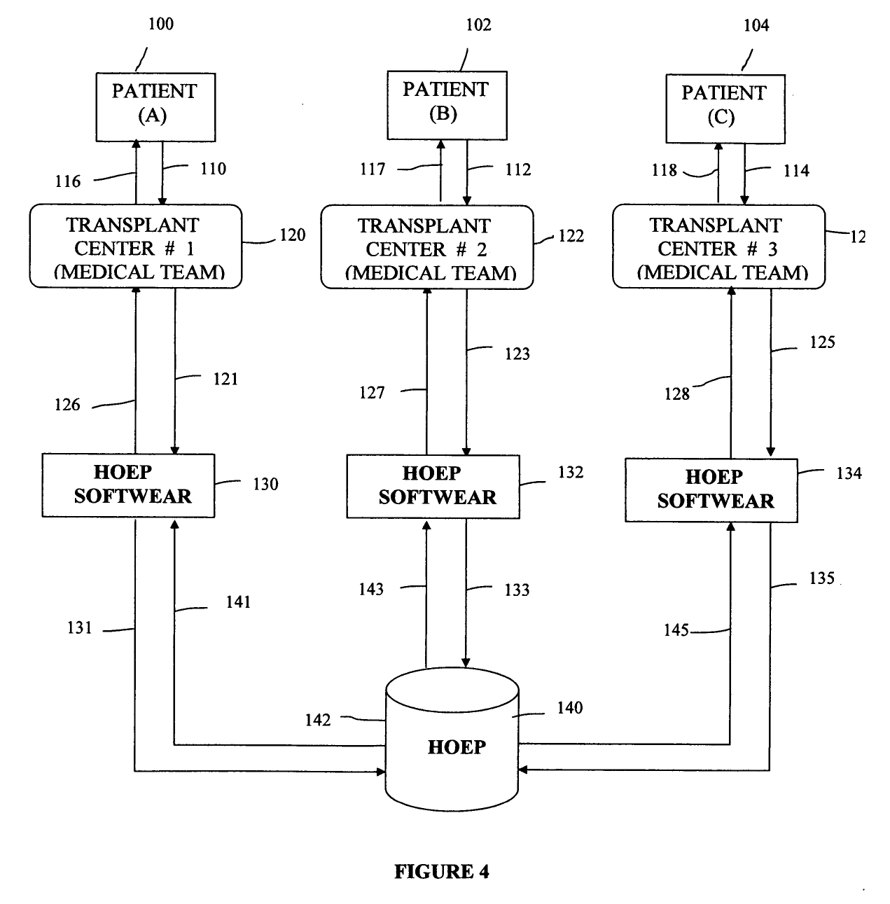

FIG. 4 is a diagrammatic depiction of the network to be used to implement the PROGRAM. In FIG. 4, Patients (A) 100, (B) 102, and (C) 104, all in need of an organ transplantation, communicate with and are evaluated by medical teams at transplantation centers #1 120, #2 122, and #3 124, respectively, by personal visitation or other communication routes, such as telemedicine, 110, 112, and 114, respectively. Each patient is evaluated for the compatibility parameters and matching criteria including the organ or part of organ which is needed and the organ or part of organ or organs which can be donated. The compatibility parameters and matching criteria are entered into computers 130, 132, and 134 using PROGRAM software at each transplantation center, 120, 122, and 124, respectively, via the data communication conduits 127, 123, and 125, respectively. A donor-recipient record is created for each patient A, B, and C. Each transplantation center computer 130, 132, and 134, using PROGRAM software, stores and communicates the donor-recipient records to a PROGRAM database 140 located in one or more server computers 142. Communication between the transplantation center computers and server computer or computers 142 via a worldwide computer network, telephone lines, wireless communications, or other data communication conduits 131, 133, 135, respectively.

The PROGRAM software enters and stores the compatibility parameter and matching criteria (donor-recipient record) for patients A 100, B 102, and C 104, into the PROGRAM database 140 in the PROGRAM server 142. Transplant or transplantation centers include hospitals, organ transplantation program, and organ procurement organizations. The transplant center computers 130, 132, and 134, using PROGRAM software, then search the PROGRAM database 140 and data communication conduits 141, 143, and 145, respectively, determines the matching of patients A 100, B 102, and C 104, respectively, with each other and with all other patients' donor-recipient records in the database 140. Information on the matches is communicated from the transplant center computers 130, 132, and 134 to the respective transplantation center medical teams #1 120, #2 122, and #3 124 via the data communication conduits 126, 127, and 128, respectively. There may be multiple medical teams which are dispersed throughout the transplantation centers. Communication between the transplant center computers and the medical teams uses convention communication means such as telephones, hard wired computer terminals, PDAs, pocket personal computers or wireless communications. Technical development in the area of communication conduits and routes is very rapid and it is anticipated that other suitable communication conduits will be developed and implemented. Information on the matches is communicated from the transplantation center medical teams #1 120, #2 122, and #3 124 to patients A 100, B 102, and 104, respectively, via communication routs 116, 117, and 118 respectively.

Alternatively, the PROGRAM server 142 searches the PROGRAM database 140 using PROGRAM software and determines the matching of patients A 100, B 102, and C 104 with each other and all other patient's donor-recipient records. Information on the matches is communicated from the PROGRAM server 142 to the respective transplantation center computers 130, 132, and 134 via the data communication conduits 141, 143, and 145, respectively. Information on the matches is communicated from the transplant center computers 130, 132, and 134 to the respective transplantation center medical teams #1 120, #2 122, and #3 124 via the data communication conduits 126, 127, and 128, respectively. Information on the matches is communicated from the transplantation center medical teams #1 120, #2 122, and #3 124 to patients A 100, B 102, and 104, respectively, via communication routes 116, 117, and 118 respectively. Although FIG. 4 shows three patients, the process may be used for an unlimited number of patients.

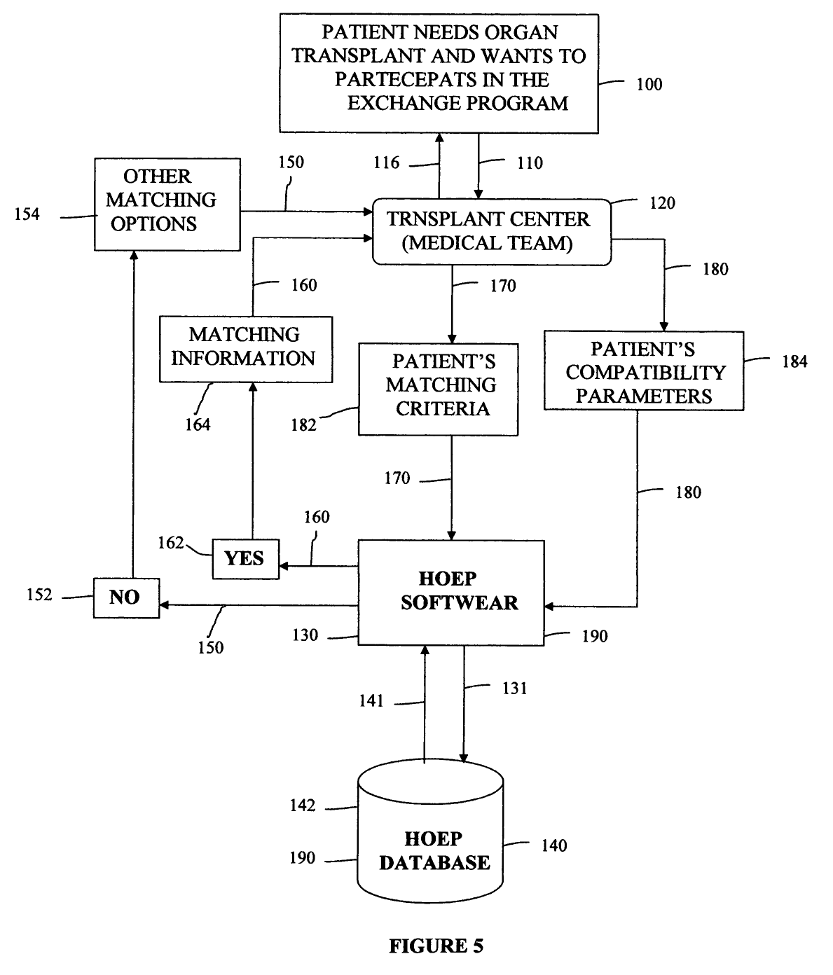

FIG. 5 is a flow chart of the processes to be used in creating a donor-recipient record, entering the record into the database, and searching the database for matches. Patient 100, in need of an organ transplantation, communicate with and are evaluated by medical teams at transplantation center 120 by personal visitation or other communication route 110. The patient is evaluated for willingness to engage in the PROGRAM, the compatibility parameters, and matching criteria including the organ or part of organ which is needed and the organ or part of organ or organs which can be donated. Data comprising the compatibility parameters 184 are entered into the transplantation center computer comprising PROGRAM software 130 via communication conduit 180 and data comprising matching criteria 182 are entered into the transplantation center computer comprising PROGRAM software 130 via communication conduit 170. The data comprising compatibility parameters 184 and data comprising matching criteria 182 comprise a donor-recipient record for patient 100 termed the requesting donor-recipient record 190. The transplantation center computer comprising PROGRAM software 130 communicates the requesting donor-recipient record 190 to a PROGRAM database 140 located in a server computerl42 via a worldwide computer network, telephone lines, or other data communication conduits 131.

The PROGRAM software enters and stores the requesting patient donor-recipient record 190 into the PROGRAM database 140 located in the PROGRAM server 142. The transplant center computer 130, using PROGRAM software, searches the PROGRAM database 140 with communication between the transplant center computer 130 and database server 142 via data communication conduits 131, and 141. The transplant center computer 130, using PROGRAM software, determines the possible matching of the requesting donor-recipient record 190 of patient 100 with all other donor-recipient records in the database 140.

If a suitable match between patient donor-recipient records is found 162, the matched donor-recipient record or records 164 are communicated via communication conduit 160 to the transplant center 120. Information on the matches is communicated from the transplantation center medical teams 120 to patient 100 via communication routes 116.

If no suitable match between the requesting donor-recipient record 190 and all other donor-recipient records in the database 140, information on the lack of match 152 is communicated to the transplant center 120 via communication conduit 150. In addition, other matching option information 154 on donor-recipient records in the database which do not match the requesting donor-recipient record but have some data in common with the requesting donor-recipient record are communicated to the transplant center 120 via communication conduit 150. Information on the lack of matches 152 and on other matching options 154 is communicated from the transplantation center medical teams 120 to patient 100 via communication routes 116.

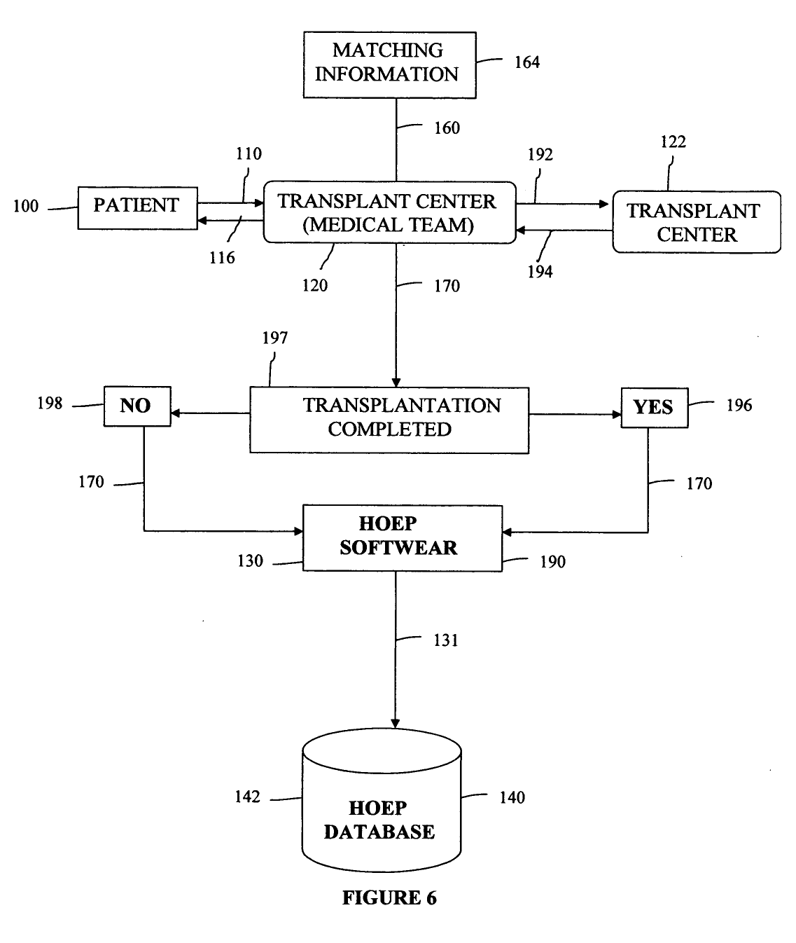

FIG. 6 is a flow chart of the processes to be used after a suitable exchange is identified. A suitable match between donor-recipients is identified from the PROGRAM database. The matching donor-recipient record or records 164 are transmitted to the requesting transplant center 120 via the data communication conduit 160. The requesting transplant center 120 communicates this information with the patient 100 via established communication route 116. If the requesting patient 100 and requesting transplant center 120 wishes to pursue the transplant, the requesting transplant center 120 communicates with the transplant center 122 (or centers if multiple matches are identified) which represents the patient having a matching donor-recipient record via communication conduits 192 and 194. Although two Transplantation Centers are shown in FIG. 6, there may be three or more Transplantation Centers involved in an exchange.

Information concerning the transplantation 197 is communicated by the requesting transplant center to the transplant center computer 130 comprising PROGRAM software 190 via communication conduit 170. If the transplantation is completed 196 the involved donor-recipient records are amended to reflect the event and the records are removed from the searchable waiting list database 140.

If, for any reason, the transplantation is not completed 198 the involved donor-recipient records are modified to reflect these facts and the involved donor-recipient records are retained in the searchable wait list database 140.

The information concerning the transplantation is communicated from the requesting transplant center computer 130 to the PROGRAM database 140 in the PROGRAM server 142 via a communication conduit 131.

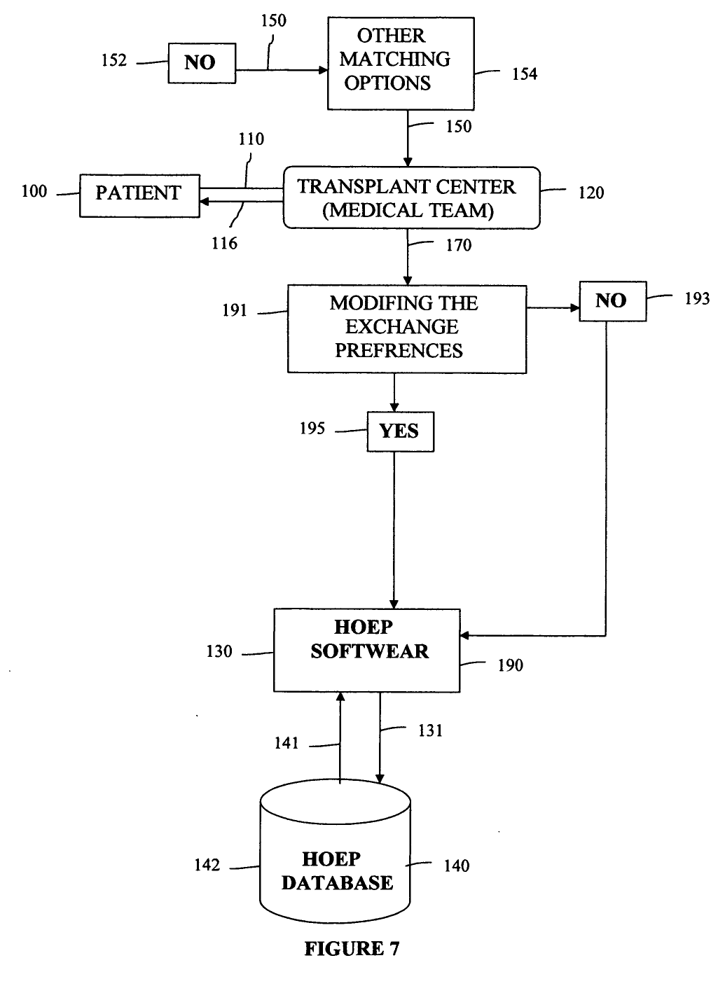

FIG. 7 is a flow chart of the processes to be used when no suitable exchange is identified using the PROGRAM database. This fact 152 is conveyed to the requesting transplant center 120, along with other matching options 154, possible matches which would be possible if the compatibility parameters and or the matching criteria were modified and made less stringent, via communication conduit 150. The transplant center 120 and the requesting patient 100 communicate via communication routes 116 and 110. They decide whether to modify the compatibility parameters and matching criteria with acceptance of the possibility of increased underlying risks.

If the requesting transplant center 120 and requesting patient 100 decide to modify 195 the compatibility parameters and or the matching criteria, the modified donor-recipient record is communicated from the requesting transplant center computer 130 to the PROGRAM database 140 in the PROGRAM server 142 via a communication conduit 131. The requesting donor-recipient record is replaced by the modified donor-recipient record and the database 140 is searched again for a match with the modified donor-recipient record.

Any patient having a donor-recipient record in the registry can at any time modify his or her donor-recipient record using the processes in the above two paragraphs.

If the transplant center 120 and patient 100 decide not to modify the requesting donor-recipient record 193, the requesting donor-recipient record is retained in the wait list database 140 for future matches with other donor-recipient records.

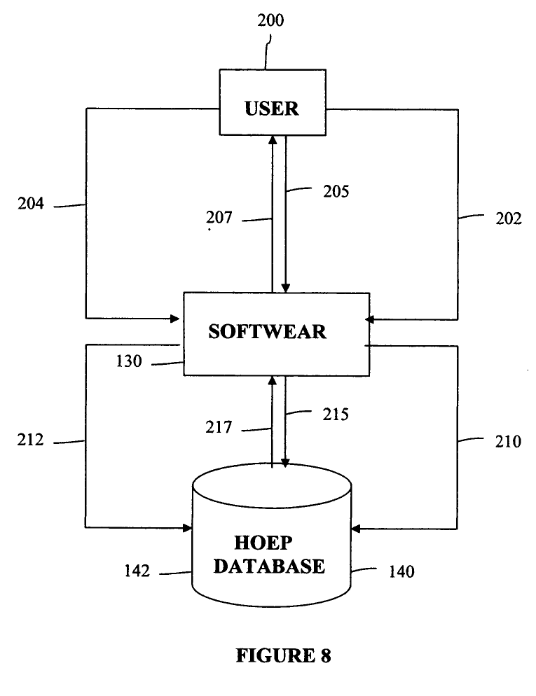

FIG. 8 is a flow chart of the relationships between users at a transplantation center, PROGRAM software, and the database. The PROGRAM system includes software and a database. In FIG. 8, the user 200 are the members of the transplantation team at a transplantation center and any other person authorized for access to the PROGRAM database, such as persons involved in scientific studies of the PROGRAM processes. The users enter 202 data regarding donor-recipients, including donor-recipient records, and information concerning the transplantation center and the medical personal at the transplantation center into the transplantation center computer 130 using the PROGRAM software. Modification of the above data also is entered 204. The user 200 enters specific instructions 205 for use of the data, such as entry into the waiting list, or searching the waiting list using the software. The transplantation center computer 130 transfers 210 the data from the transplantation center computer to the server 142 to be entered into and stored in the database 140. Modifications to the data stored in the database 140 are transferred 212 by the transplantation center computer 130. The transplantation center computer 130 transfers specific instructions 215 for use of the data, such as searching the database for matches between donor-recipient records. Results of the instructions for use of the data are transferred 217 from the database 140 and server 142 to the transplantation center computer 130. The transplantation center computer 130 transfers 207 the results to the user 200.

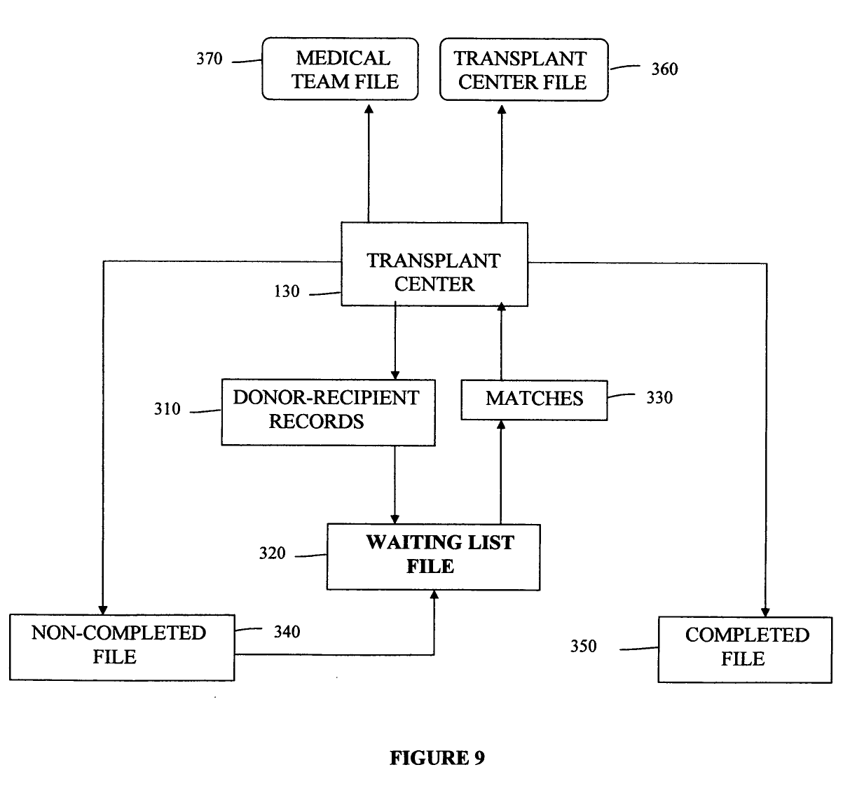

FIG. 9 is a flow chart showing the flow of information between files comprising the registry in the PROGRAM database and the transplantation center 130. The PROGRAM registry or database located in the PROGRAM server comprises at least five files. The waiting list file is a collection of donor-recipient records of patients waiting for a suitable match for transplantation. The waiting list file also includes the donor-recipient records of patients who have already undergone an organ exchange, but who are in need of an additional organ donation. Donor-recipient records 310 and modifications to donor-recipient records are sent from the transplantation centers 130 to the waiting list 320 file. Matches 330 between donor-recipient records identified by searches are communicated to the transplantation centers. The completed exchange 350 file contains information concerning completed PROGRAM organ exchanges which is provided by the transplantation centers, and includes information on the condition of the involved patients. The non-completed 340 file contains information on matches which were identified from the wait list but, for whatever reason, did not result in an exchange of organs. Donor-recipient records in the non-completed file 340 are also entered in the waiting list 320 file. The transplantation center 360 file contains information concerning the transplantation centers and their experience with the PROGRAM program. The medical team 370 file contains information concerning the physicians and medical teams involved in the PROGRAM process which includes current positions and experience with transplantation.

It is anticipated that other files will be added to the database as appropriate. Each file is periodically modified to include the latest information concerning the participants in the PROGRAM system. It is anticipated that the files will be modified to reflect the deaths of patients, desire to be removed from the waiting list, changes in venue of the medical team members, and other relevant information. The files will provide valuable information for scientific study of the PROGRAM process. Information on the individuals involved will be released and used only in compliance with all laws.

The patients and transplant centers typically located dispersed throughout a country to facilitate visits by the patient to the transplant center. The transplant centers can be in any country. The PROGRAM server and database may be in a single location, but also may be located in a number of locations.

Any suitable personal computer with adequate speed and capacity may be used as a transplantation center computer.

Any suitable server computer with adequate speed and storage capacity may be used as a computer for storing and, if desired, searching the database comprising the human organ exchange program registry.

Any suitable software which is used for creating databases and for searching databases may be used for the PROGRAM software located at the transplant centers. Any suitable software used for storing and searching large numbers of records in a database may be used for the PROGRAM software located at the database server. A preferred software is the PROGRAM software which executes the processes shown on FIGS. 8 and 9 and disclosed in detail in the discussion of FIGS. 8 and 9 above.

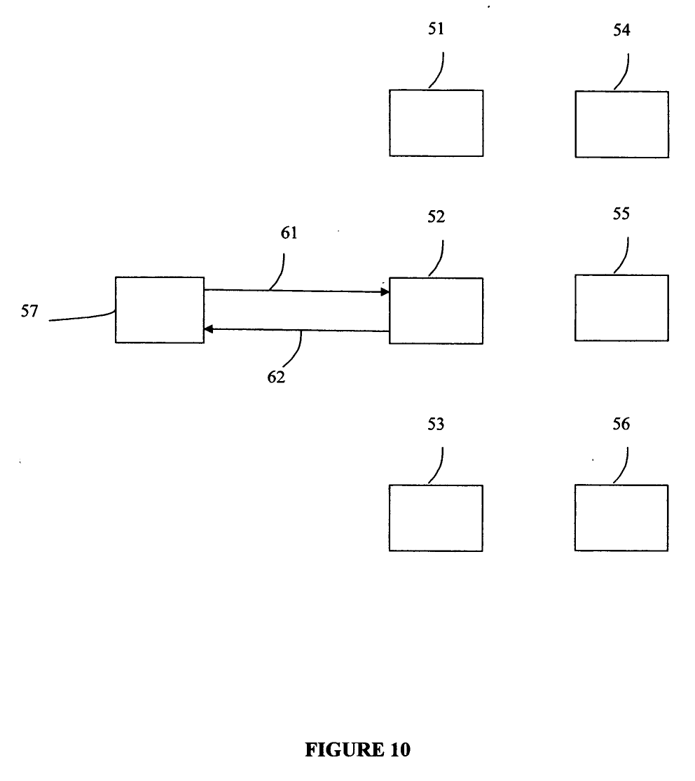

FIG. 10 is a diagrammatic depiction of an exchange of organs between a living donor and a recipient followed by receipt of an organ from the recipient after death. Donor-recipient members of the registry are shown at 51, 52, 53, 54, 55, 56, and 57. Member 57 is capable of donating an organ, for example a kidney, and has need of a liver. Member 57 has made the donation 61 of an organ, for example a kidney, to member 52 who is in need of a kidney. Member 52 is unable to donate an organ because of illness. On his or her death, member 52 donates an organ 62, for example a liver, to member 57, who is in need of a liver. In the third embodiment, membership in the donor-recipient registry carries the opportunity to receive a needed organ from another member of the registry. Membership in the registry also carries the obligation to donate an organ to another member of the registry, while the donor is alive or after death of the donor.

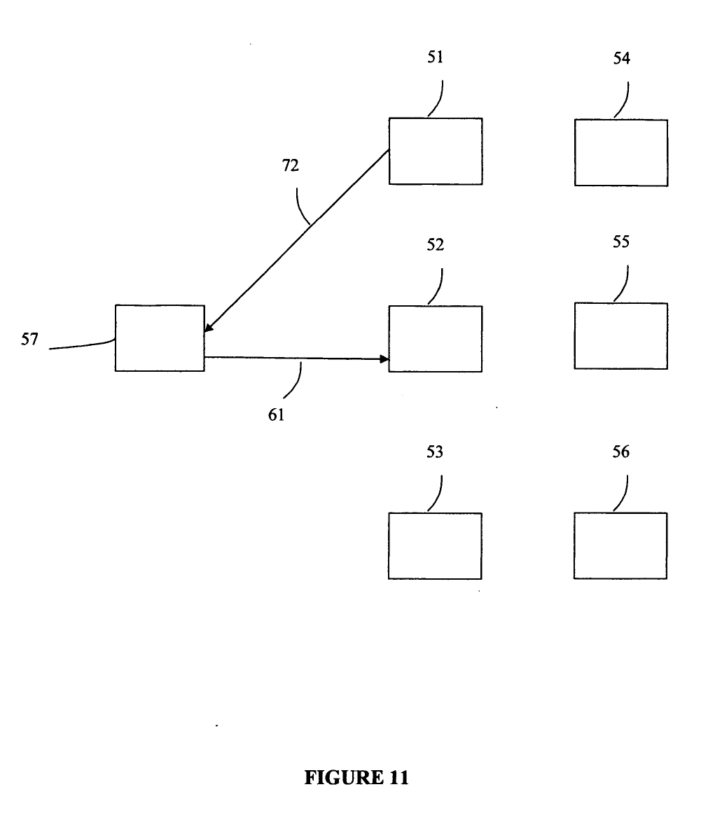

FIG. 11 is a diagrammatic depiction of an exchange of organs between a living donor and a recipient followed by receipt of an organ from a different member of the donor-recipient registry after death. FIG. 11 is the same as FIG. 10, except member 52 is unable to donate an organ even after death, but another member 51 of the registry after death donates an organ 72, such as a liver, to member 57, who is in need of a liver.

It will be apparent to those skilled in the art that the examples and embodiments described herein are by way of illustration and not of limitation, and that other examples may be used without departing from the spirit and scope of the present invention, as set forth in the appended claims.

1. A method for creating and maintaining a human organ exchange registry containing records of donor-recipient patients in need of an organ transplant and capable of and willing to donate an organ other than the one for which they have a need to another donor-recipient in need of an organ transplant and capable of and willing to donate an organ comprising the steps:
a. creating a new donor-recipient record for a potential donor-recipient comprising compatibility parameters and matching criteria including the organ or part of organ needed by the potential donor-recipient, and the organ or part of organ which can be donated by the potential donor-recipient to another potential donor-recipient using suitable software,
b. storing the donor-recipient record in a waiting list file in a database for access by other donor-recipients using suitable software, and
c. modifing the donor-recipient record as the donor-recipient's circumstances change using suitable software.

2. The method of claim 1 wherein step a. further comprises information on the size of the organ or part of the needed organ.

3. The method of claim 1 wherein step a. further comprises information on the time transplantation is desired.

4. The method of claim 1 wherein step a. is performed at a transplant center and step b. is performed at a database server.

5. The method of claim 1 further comprising the step after step b.:
b1. searching the database for matches between each new donor-recipient record and donor-recipient records in the database using suitable software,

6. The method of claim 5 wherein the database is searched using a computer comprising suitable software located at the transplant center.

7. The method of claim 5 wherein the database is searched using a computer comprising suitable software located at the database server computer.

8. The method of claim 5 when no match between donor-recipient records is found, further comprising the step after step b1:
b2: modifying the compatibility parameters or matching criteria of the new or existing donor-recipient record,

9. The method of claim 5 when a match between donor-recipient records is found, further comprising the steps after step b1:
b2: creating a completed file if an exchange of organs takes place between patients with matched donor-recipient records, the completed file to comprise matched donor-recipient records and the outcome of the exchange,
b3: creating a non-completed file if no exchange of organs takes place between patients with matched donor-recipient records, the non-completed file to comprise matched donor-recipient records and the reasons for non-completion of the exchange of organs, and
b4: reentering the matched donor-recipient records in the non-completed file into the waiting list file,

10. The method of claim 1 wherein the human organ exchange registry further comprises a transplantation center list comprising centers where transplantations take place at the transplantation centers comprising the steps;
d. modifying the transplantation center list when a transplantation between donor-recipients takes place at a transplantation center with information on the transplantation and the outcome of the transplantation.

11. The method of claim 1 wherein the human organ exchange registry further comprises a medical team list comprising medical personnel at the transplantation centers where transplantations take place comprising the step;
d. modifying the medical team list with information concerning the experience of the medical team and current location of the medical personnel.

12. The method of exchanging donatable human organs between a first person with a need for a donatable human organ transplant and a second person with a need for a donatable human organ transplant, the donatable organ needed by the second person differing from the donatable organ needed by the first person, comprising the steps:
a. transplanting a donatable human organ needed by the first person from the second person to the first person, and
b. transplanting a donatable human organ differing from the donatable organ needed by the first person which is needed by the second person from the first person to the second person.

13. The method of exchanging donatable human organs between a first person with a need for a donatable human organ transplant and a second person with a need for a donatable human organ transplant, the donatable organ needed by the second person differing from the donatable organ needed by the first person, and a third person with a need for a donatable human organ transplant, the donatable organ needed by the third person differing from the donatable organs needed by both the first and second persons comprising the steps:
a. transplanting a donatable human organ needed by the second person from the first person to the second person, and
b. transplanting a donatable human organ differing from the donatable organs needed by the first person and the second person which is needed by the third person from the second person to the third person, and
c. transplanting a donatable human organ differing from the donatable organs needed by the second person and the third person which is needed by the first person from the third person to the first person.

14. The method of exchanging donatable human organs between N persons with a need for a human donatable organ transplant comprising the steps:
a. transplanting a donatable human organ needed by a first person from a second person to the second person, wherein the donatable human organ needed by the first person differs from that needed by the second person, and
b. repeating step a. wherein the second person is termed the first person and a third person is termed the second person, until each of the N persons has received the needed donatable human organ and each of the N persons has donated a donatable human organ other than the organ needed by that person.

15. The process of exchanging a donatable human organ between a living first person in need of a human donatable organ transplant and a living second person in need of a human donatable organ transplant who is willing to donate an organ to the first person, or between the first person and a deceased third person who, while living, was willing to donate an organ to the first person, comprising the steps,
a. forming a donor-recipient registry of persons in need of a donatable human organ who are willing to donate a different human donatable organ to another person on the registry while the donor is alive or after death of the donor, and
b. transplanting a donatable human organ to a living first person who is listed on the registry from a living second person who is listed on the registry or from a deceased third person who is listed on the registry.

16. The method of claim 12 wherein the transplantation of the organ of step b occurs after the death of the first person.

17. The method of claim 13 wherein the transplantation of the organ of step b occurs after the death of the third person.

18. The method of claim 14 wherein the transplantation of the organ of step b occurs after the death of the last person to donate an organ.
